(19) United States
(12) Patent Application Publication
(10) Pub. No.: US 2017/0347992 A1
(43) Pub. Date: Dec. 7, 2017

(54) AUTOMATED REGION OF INTEREST PLACEMENT

(71) Applicant: Carestream Health, Inc., Rochester, NY (US)

(72) Inventor: Ajay Anand, Rochester, NY (US)

(21) Appl. No.: 15/268,889

(22) Filed: Sep. 19, 2016

Related U.S. Application Data
(60) Provisional application No. 62/344,466, filed on Jun. 2, 2016.

(51) Int. Cl. A61B 8/08; A61B 8/00

(52) U.S. Cl. CPC A61B 8/5215; A61B 8/488; A61B 8/463; A61B 8/485

(57) ABSTRACT

A method displays an ultrasound image of subject tissue that defines a region of interest, the image acquired in a first measurement mode. In response to an operator instruction, the method switches to a second measurement mode, analyzes the subject tissue within the region of interest for compatibility with the second measurement mode, and highlights one or more areas of the subject tissue for removal from the region of interest, displaying a revised region of interest according to the mode compatibility analysis. One or more signals for the second measurement mode are directed to the revised region of interest and measurement results displayed.

### CROSS REFERENCE TO RELATED APPLICATIONS

The present application claims the benefit of U.S. Provisional application U.S. Ser. No. 62/344,466, provisionally filed on 2 Jun. 2016 entitled “AUTOMATED REGION OF INTEREST PLACEMENT”, in the names of Ajay Anand, all of which are incorporated herein in their entirety.

### FIELD OF THE INVENTION

The invention relates generally to medical ultrasound systems and methods, and in particular to a method for improved workflow for ultrasound apparatus operation.

### BACKGROUND

Ultrasound imaging systems/methods are known, such as those described, for example, in U.S. Pat. No. 6,705,995 (Poland), U.S. Pat. No. 5,370,120 (Oppelt), and U.S. Pat. No. 8,285,357 (Gardner), all of which are incorporated herein in their entirety. Various applications for diagnostic ultrasound systems are given, for example, in the article entitled “Ultrasound Transducer Selection In Clinical Imaging Practice”, by Szabo and Lewin, Journal of Ultrasound Medicine, 2013; 32:573-582, incorporated herein by reference in its entirety.

Ultrasound utilizes sound waves at frequencies higher than those perceptible to the human ear. Ultrasonic images known as sonograms are generated as a result of pulsed ultrasonic energy that has been directed into tissue using a probe. The probe obtains echoed sound energy from the internal tissue and provides signal content that represents the different sound reflectivity exhibited by different tissue types. This signal content is then used to form images that visualize features of the internal tissue. Medical ultrasound, also known as diagnostic sonography or ultrasonography, is used as a diagnostic imaging technique used to help visualize features and operation of tendons, muscles, joints, vessels and internal organs of a patient.

FIGS. 1A-1B and FIGS. 2-3 show exemplary portable ultrasound systems 10 that use a cart/base/support, cart 12, a display/monitor 14, one or more input interface devices 16 (such as keyboard or mouse), and a generator 18. The display/monitor 14 can also be a touchscreen to function as an input device. As illustrated, the ultrasound system 10 can be a mobile or portable system designed to be wheeled from one location to another. As FIG. 2 shows, the ultrasound system 10 has a central processing unit CPU 20 that provides control signals and processing capabilities. CPU 20 is in signal communication with display 14 and interface device 16, as well as with a storage device 22 and an optional printer 24. A transducer probe 26 provides the ultrasound acoustic signal and generates an electronic feedback signal indicative of tissue characteristics according to the echoed sound.

FIG. 3 shows an example of an ultrasound system 10 in use with an image provided on display/monitor 14.

Different types of images, with different appearance, can be formed using sonographic apparatus. The familiar monochrome B-mode image displays the acoustic impedance of a two-dimensional cross-section of tissue. Other types of image can use color or other types of highlighting to display specialized information such as blood flow, motion of tissue over time, the location of blood, the presence of specific molecules, tissue stiffness, or the anatomy of a three-dimensional region.

Accordingly, the ultrasound systems of FIGS. 1A-3 are typically configured to operate within at least two different ultrasound modes. As such, the system provides means to switch between the at least two different ultrasound modes. Such a multi-mode configuration, along with techniques for switching between modes, are known to those skilled in ultrasound technology.

In conventional workflow, the sonographer or other operating practitioner begins an examination with B-mode imaging in order to locate the anatomy or region of interest (ROI). B-mode imaging is relatively unconstrained, providing at least sufficient information for identifying prominent anatomical features. Then, once the ROI is located, the sonographer switches to a suitable imaging mode for the particular requirements of an exam, in which more specialized signals and signal sensing may be used. In switching from one mode to the next, however, the sonographer must often readjust various equipment settings and may need to manually identify or adjust the ROI for the new mode. For example, there can be portions of the ROI that either require special imaging treatment or that simply can't be acceptably imaged using a particular mode. The need for this type of tedious and repeated adjustment complicates sonographer workflow, adding time and steps to the procedure to obtain the desired image. In some instances, a more experienced sonographer may be familiar with limitations of an imaging mode and can anticipate and avoid problems that would otherwise tend to confuse or complicate the imaging task for a less experienced technician.

Accordingly, there is a desire to provide improved workflow for the Sonographer and to improve workflow and address problems that can result from changing the ultrasound equipment mode.

### SUMMARY

According to one aspect of the invention, there is provided a system and method for ultrasound imaging. An object of the present disclosure is to advance the art of ultrasound imaging and to provide a method and apparatus that can automate definition of the region of interest for ultrasound imaging modes.

These aspects are given only by way of illustrative example, and such objects may be exemplary of one or more embodiments of the invention. Other desirable objectives and advantages inherently achieved by the disclosed invention may occur or become apparent to those skilled in the art. The invention is defined by the appended claims.

According to an embodiment of the present disclosure, there is provided a method comprising: displaying an ultrasound image of subject tissue that defines a region of interest, the image acquired in a first measurement mode; in response to an operator instruction: (i) switching to a second measurement mode; (ii) analyzing the subject tissue within the region of interest for compatibility with the second measurement mode; (iii) highlighting one or more areas of the subject tissue for removal from the region of interest and displaying a revised region of interest according to the mode compatibility analysis; directing one or more signals for the second measurement mode to the revised region of interest; and displaying measurement results.

### BRIEF DESCRIPTION OF THE DRAWINGS

The foregoing and other objects, features, and advantages of the invention will be apparent from the following more particular description of the embodiments of the invention, as illustrated in the accompanying drawings. The elements of the drawings are not necessarily to scale relative to each other.

FIGS. 1A and 1B show exemplary ultrasound systems.

FIG. 2 shows a schematic of an exemplary ultrasound system.

FIG. 3 illustrates a sonographer using an exemplary ultrasound system.

FIG. 4 shows a displayed ultrasound image having a default region of interest, shown in grayscale.

FIG. 5 shows a displayed ultrasound image having a region of interest shown as a bounded rectangle, wherein features within the region of interest are highlighted in color.

FIG. 6A is a schematic diagram that shows an initial ROI obtained using a survey mode.

FIG. 6B is a schematic diagram showing an ROI adjustment that is used in switching from a survey mode to a functional mode.

FIG. 7A shows an exemplary ROI that includes a patient's liver.

FIG. 7B shows regions of the original ROI that are not imaged in a satisfactory manner using a particular functional mode.

FIG. 8 shows a logic flow diagram of a procedure for obtaining images in a sequence of modes according to the method of the present disclosure.

### DETAILED DESCRIPTION OF EXEMPLARY EMBODIMENTS

The following is a detailed description of the embodiments of the invention, reference being made to the drawings in which the same reference numerals identify the same elements of structure in each of the several figures.

As used herein, the term “energizable” relates to a device or set of components that perform an indicated function upon receiving power and, optionally, upon receiving an enabling signal.

In the context of the present disclosure, the phrase “in signal communication” indicates that two or more devices and/or components are capable of communicating with each other via signals that travel over some type of signal path. Signal communication may be wired or wireless. The signals may be communication, power, data, or energy signals. The signal paths may include physical, electrical, magnetic, electromagnetic, optical, wired, and/or wireless connections between the first device and/or component and second device and/or component. The signal paths may also include additional devices and/or components between the first device and/or component and second device and/or component.

In the context of the present disclosure, the term “subject” or “body” or “anatomy” is used to describe a portion of the patient that is undergoing ultrasound imaging. The terms “sonographer”, “technician”, “viewer”, “operator”, and “practitioner” are used to broadly indicate the person who actively operates the sonography equipment.

The term “highlighting” for a displayed element or feature has its conventional meaning as is understood to those skilled in the information and image display arts. In general, highlighting uses some form of localized display enhancement to attract the attention of the viewer. Highlighting a portion of a display, such as a particular value, graph, message, or other element can be achieved in any of a number of ways, including, but not limited to, annotating, displaying a nearby or overlaying symbol, outlining or tracing, display in a different color or at a markedly different intensity or grayscale value than other image or information content, blinking or animation of a portion of a display, or display at larger scale, higher sharpness, or contrast.

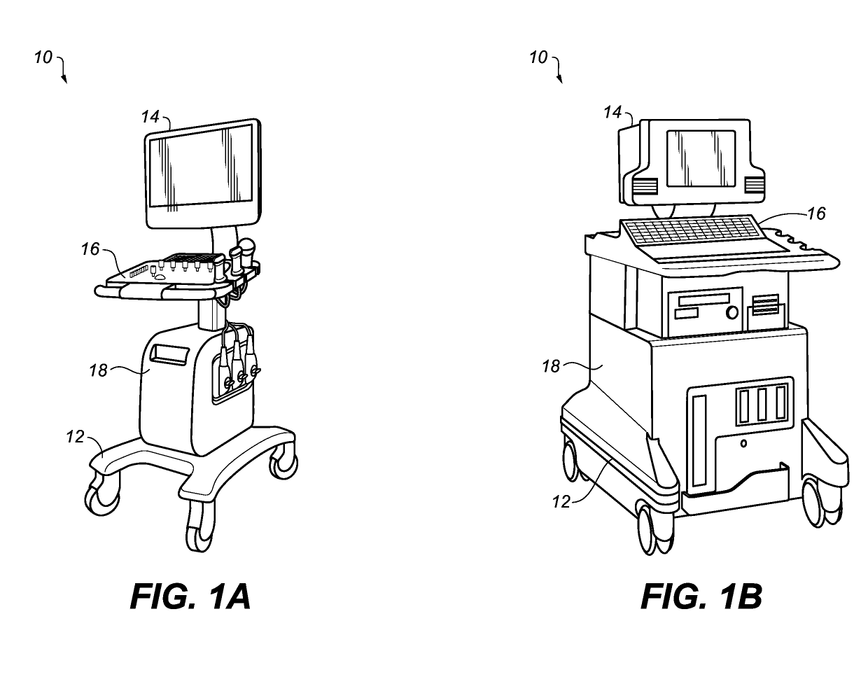

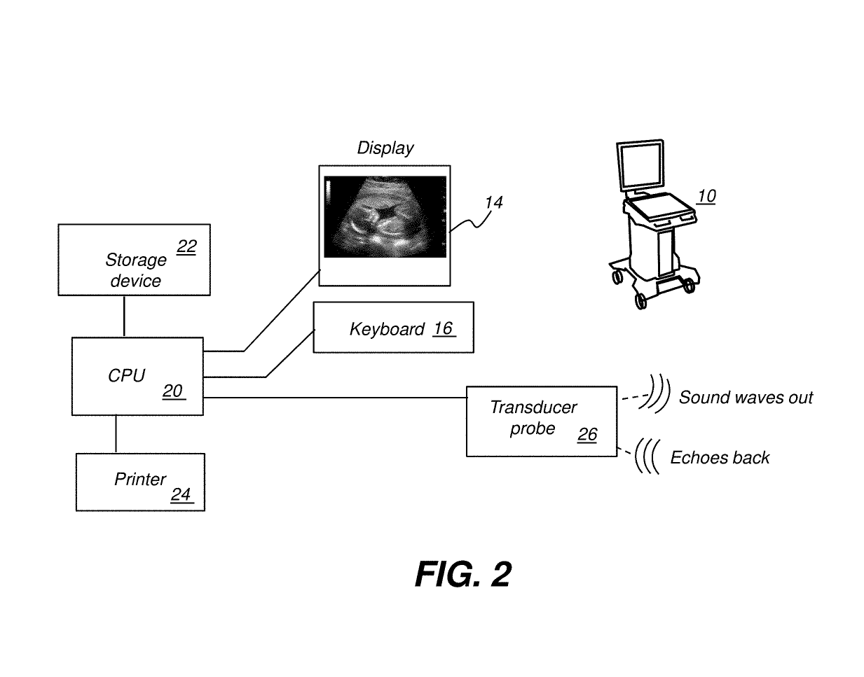

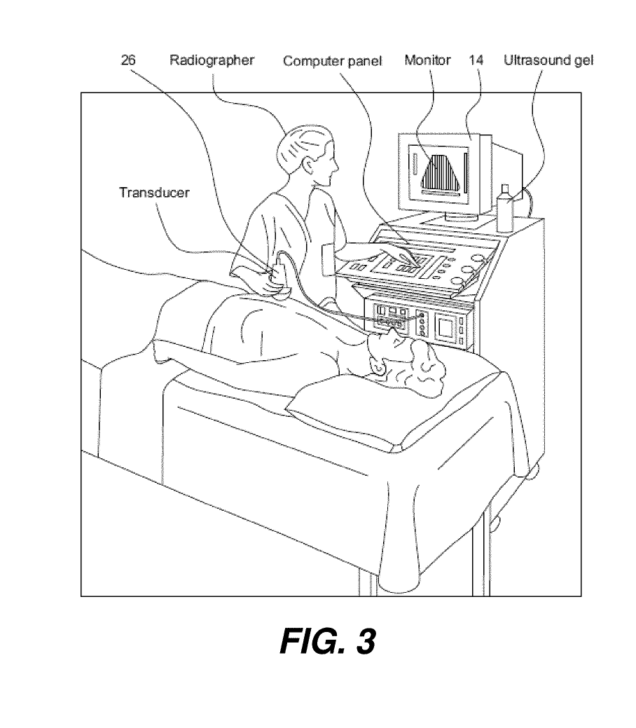

The ultrasound system, shown by way of example in FIGS. 1A and 1B, can include image processing system, a user interface and a display. The image processing system includes a memory and a processor. Additional, different or fewer components may be provided in the system or image processing system. In one embodiment, the system is a medical diagnostic ultrasound imaging system. The memory is a RAM, ROM, hard drive, removable media, compact disc, DVD, floppy disc, tape, cache memory, buffer, capacitor, combinations thereof or any other now known or later developed analog or digital device for storing information. The memory is operable to store data identifying a selected point for identifying a region of interest. The memory is operable to store data identifying one or a plurality of region of interest. Information from the user interface indicating a position on an image on the display is used to determine a spatial relationship of a user selected point to a scanned region or image position. The selected point is an individual or single point in one embodiment that may be a point selected within a line, area or volume. Additional or different information may be also stored within the memory. The processor is general processor, application specific integrated circuit, digital signal processor, controller, field programmable gate array, digital device, analog device, transistors, combinations thereof or other now known or later developed devices for receiving analog or digital data and outputting altered or calculated data. The user input is a track ball, mouse, joy stick, touch pad, buttons, slider, knobs, position sensor, combinations thereof or other now known or later developed input devices. The user input is operable to receive a selected point from a user. For example, the user positions a cursor on an image displayed on the display. The user then selects a position of the cursor as indicating a point for a region of interest. The display is a CRT, LCD, plasma screen, projector, combinations thereof or other now known or later developed devices for displaying an image, a region of interest, region of interest information and/or user input information.

Modes of ultrasound used in medical imaging include the following:

A-mode: A-mode (amplitude mode) is the simplest type of ultrasound. A single transducer scans a line through the body with the echoes plotted on screen as a function of depth. Therapeutic ultrasound aimed at a specific tumor or calculus also uses A-mode emission to allow for pinpoint accurate focus of the destructive wave energy.

B-mode or 2D mode: In B-mode (brightness mode) ultrasound, a linear array of transducers simultaneously scans a plane through the body that can be viewed as a two-dimensional image on screen. Sometimes referred to as 2D mode, this mode is effective for showing positional and dimensional characteristics of internal structures and is generally the starting point for exam types that use other modes.

C-mode: A C-mode image is formed in a plane normal to a B-mode image. A gate that selects data from a specific depth from an A-mode line is used; the transducer is moved in the 2D plane to sample the entire region at this fixed depth. When the transducer traverses the area in a spiral, an area of 100 cm² can be scanned in around 10 seconds.

M-mode: In M-mode (motion mode) ultrasound, pulses are emitted in quick succession. With each pulse, either an A-mode or B-mode image is acquired. Over time, M-mode imaging is analogous to recording a video in ultrasound. As the organ boundaries that produce reflections move relative to the probe, this mode can be used to determine the velocity of specific organ structures.

Doppler mode: This mode makes use of the Doppler effect in measuring and visualizing blood flow.

Color Doppler: Velocity information is presented as a color-coded overlay on top of a B-mode image. This mode is sometimes referred to as Color Flow or color mode.

Continuous Doppler: Doppler information is sampled along a line through the body, and all velocities detected at each point in time are presented (on a time line).

Pulsed wave (PW) Doppler: Doppler information is sampled from only a small sample volume (defined in 2D image), and presented on a timeline.

Duplex: a common name for the simultaneous presentation of 2D and (usually) PW Doppler information. (Using modern ultrasound machines, color Doppler is almost always also used; hence the alternative name Triplex.).

Pulse inversion mode: In this mode, two successive pulses with opposite sign are emitted and then subtracted from each other. This implies that any linearly responding constituent will disappear while gases with non-linear compressibility stand out. Pulse inversion may also be used in a similar manner as in Harmonic mode.

Harmonic mode: In this mode a deep penetrating fundamental frequency is emitted into the body and a harmonic overtone is detected. With this method, noise and artifacts due to reverberation and aberration are greatly reduced. Some also believe that penetration depth can be gained with improved lateral resolution; however, this is not well documented.

Elastography mode: this mode maps the elastic properties of soft tissue. Tissue response indicating hardness or softness can yield diagnostic information about the presence or status of disease. For example, cancerous tumors are often noticeably harder than the surrounding tissue, and diseased livers stiffer than healthy ones. Shear Wave Elasticity Imaging and Shear Wave Imaging are described in more detail below.

While conducting an ultrasound exam, the sonographer may often switch between multiple ultrasound modes as part of standard workflow. In conventional practice, for example, the sonographer first operates in a B-mode in order to coarsely locate the ROI and to show overall shapes and dimensions of internal features. The sonographer then transitions to a Doppler mode before moving back to the B-mode. For some particular examinations, there are pre-set (or pre-determined or pre-defined) steps and a predetermined sequence of modes that the Sonographer must follow, often beginning with B-mode imaging. That is, the ordered sequence of modes used in a particular exam type can be predefined for the operator.

For carotid artery imaging, for example, the exam typically follows a progression of modes such as the following:

(i) B-mode for initial positioning and establishing reference coordinates of the sample volume;

(ii) Color Flow mode for improved visualization of blood vessels; and

(iii) Pulse wave Doppler mode for highlighting blood flow within the sample volume.

For heart imaging, the exam progression can use B-mode or M-mode imaging for auto-positioning of the cursor, followed by Color Flow or pulse wave Doppler modes.

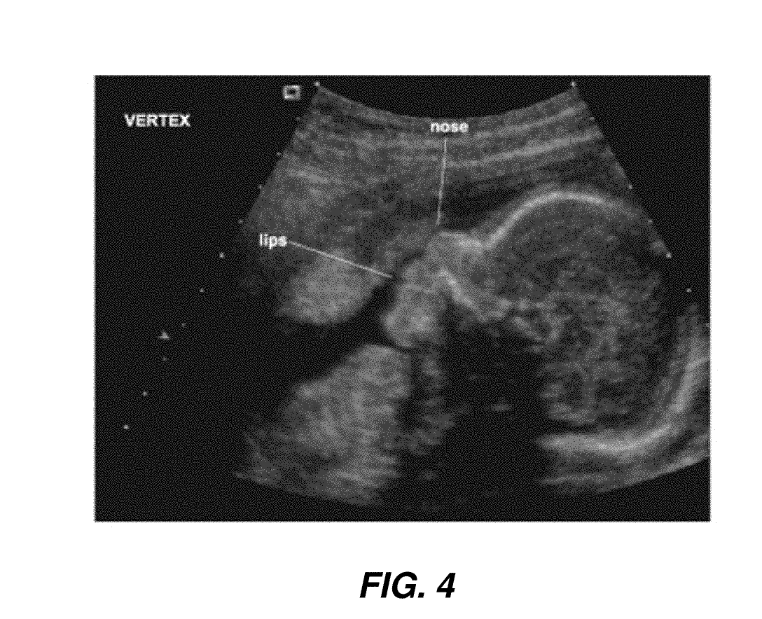

Thus, as a starting point, the sonography workflow typically begins with acquisition and display of a grayscale mode image (such as the B-mode image illustrated in FIG. 4) in order to survey and scale the anatomy. FIG. 4 shows an exemplary B-mode ultrasound image, displayed as a grayscale image.

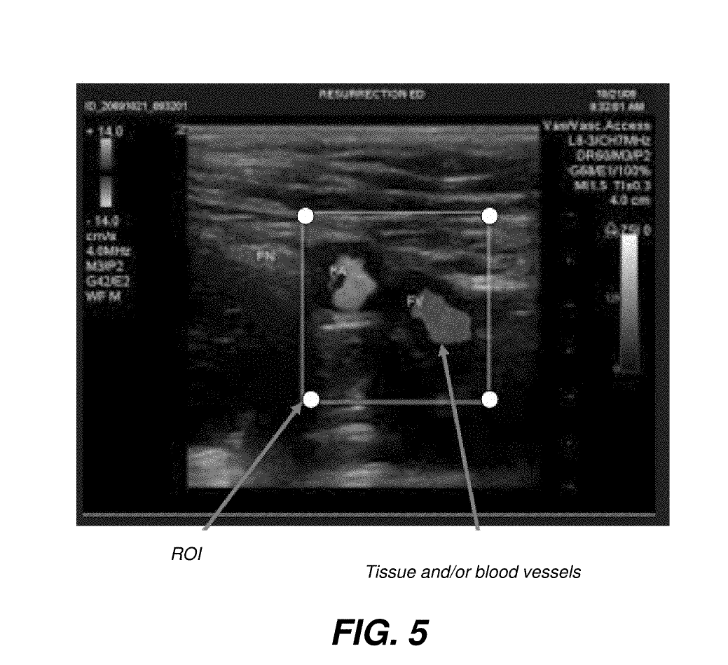

FIG. 5 shows an image with an ROI that is outlined and has color highlighting for tissue and blood vessels, obtained in Color Flow mode. When viewing an ultrasound image on the display, the particular area of the displayed image that is of interest to the sonographer or other practitioner is referred to as the Region of Interest (ROI) or, alternately, the ROI extent. As the sonographer conducts the examination and switches between imaging modes, the displayed size and position, as well as the apparent shape of the ROI may change.

The region of interest (ROI) can be defined and displayed in any of a number of ways. In conventional practice, as shown in the example of FIG. 5, the ROI is defined by multiple points or vertices that define a polygon shape, such as defining a rectangle or other parallelogram by its four corners, as shown. Alternately, the ROI can be defined by a point and a distance, such as a center point and a radius or function of the distance from the point to a single boundary. The distance may be, for example, any of a radius, circumference, diagonal, length or width, diameter or other characteristic of a shape. The region of interest can alternately be defined by a point and two distances, such as a distance to each of two boundaries, or using an angle. In another arrangement, the region of interest can be a pre-defined shape positioned around a point, such as a square, rectangle, oval or combination thereof.

An embodiment of the present disclosure describes a system and method for an automated ROI placement that can be particularly useful for Shear Wave Imaging. This automated tool provides particular features for operator assistance, including anatomical marks, markers, landmarks, visual queues/patterns, and/or visual markers.

As described previously, Shear Wave Elasticity Imaging (SWEI) refers to a sonography method for mapping tissue elasticity, a tissue characteristic measured according to dimensional or movement response to the acoustic signal. Particularly useful for soft tissue measurement, the method employs the force of acoustic radiation from focused ultrasound to generate shear waves in the soft tissue. A tissue elasticity map can be formed by measuring shear wave propagation parameters using ultrasound or MRI (Magnetic Resonance Imaging).

The terms “Elasticity Imaging” and “Elastography” are typically viewed as synonymous, thus the initialism SWEI (Shear Wave Elastography Imaging) is often shortened to SWE (Shear Wave Elastography). The shear wave speed is governed by the shear modulus of tissue which is highly sensitive to physiological and pathological structural changes of tissue. Variation of the shear modulus may range over several orders of magnitude depending on the structure and state of tissue. This variation of the shear wave speed increases in many tissues in the presence of disease, e.g. known cancerous or tissues can be significantly stiffer than normal tissue. For this reason, the possibility of using shear waves in new diagnostic methods and devices has been extensively investigated over the last two decades.

Various parameters of tissue that characterize its structure and state such as anisotropy, viscosity, and nonlinearity, can be assessed using ultrasonic shear waves. Shear waves are polarized which makes them sensitive to tissue anisotropy, which is a structural anatomical characteristic that can have diagnostic value. By directing shear waves in different directions, some practitioners believe it is possible to more accurately characterize tissue anisotropy. Following this approach, the large frequency range of the shear wave that can be generated in tissue appears to have benefit for tissue diagnostics and to offer considerable potential for characterizing tissue viscoelastic properties. Shear wave attenuation is inherently high; thus, the directed acoustic shear waves do not propagate very deeply into the subject tissue. This is viewed by some as an advantage because the shear waves induced by acoustic radiation force are less prone to artifacts from reflections and interactions along other tissue boundaries.

Applicants have recognized that Shear Wave imaging results, while they offer some promise for more effective tissue characterization, may not be conclusive when performed near or adjacent to particular anatomical structures. Factors such as heterogeneous tissue composition and boundary effects can tend to complicate sensing and interpretation of the received signal. By way of example, the liver capsule is one anatomical structure that can be particularly troublesome for this reason. In this case, for example, clinical guidelines for liver elastography recommend avoiding attempts to obtain shear wave acoustical measurements for tissue near the liver capsule. Thus, it is recommended that shear wave measurements be performed no closer than about 1.5 cm below the liver capsule. Similar constraints exist for other types of elastography measurement as well. In current practice, it is the clinical practitioner's expertise that guides where, and where not, to make elastography measurements using shear wave mode in order to obtain reliable results.

In order to more precisely define the operation of the Applicants' method, it is useful to categorize ultrasound imaging modes according to the type(s) of acoustic signal used for each mode and whether or not the sensed measurement primarily obtains static position and dimensional information or measures movement, such as fluids movement, or response to acoustical signal variation. Using these general criteria, each ultrasound can be classified as one of either of the following:

(i) Survey Modes. This category includes more static ultrasound modes that primarily show position and dimension. Survey modes broadly enable the patient anatomy that is under study to be identified, located in space, and dimensioned, and includes A-mode, B-mode, C-mode, M-mode, and harmonic mode. The survey modes can be considered as mapping modes, using acoustic energy to identify and present the overall anatomy of interest as the overall ROI. Survey mode scanning is characterized by relatively low energy levels, moderate to low computational demands with relatively straightforward computation, and generates broader areas of image content, so that the image coverage is sufficient to include the region of interest and surrounding portions of the anatomy. In conventional practice, initial measurements of the patient are obtained in a survey mode and the ROI displayed accordingly. There are no anatomy-related constraints for imaging type for typical survey modes. B-mode imaging is the predominant survey mode used in standard practice, and is considered compatible with virtually all tissue types.

(ii) Functional Modes. This category includes more specialized, dynamic imaging modes that characterize changing aspects or features of the subject tissue, including tissue response over a range of frequencies and temporal attributes such as fluid or gas flow and flow velocity. Other attributes measured using functional mode imaging can include tissue stiffness or elasticity, for example. Functional modes provided with the typical ultrasound system can include shear wave imaging SWEI, as described in more detail herein, as well as various types of Doppler imaging, including color Doppler, continuous Doppler, pulsed wave Doppler, and pulse inversion. Functional modes may not be useful over the full ROI defined by the corresponding survey modes and can be limited in some applications where they are useful, according to anatomical characteristics. Some anatomical features are considered incompatible with particular functional modes, as described earlier with reference to the liver capsule and SWEI imaging, for example.

It is noted that the identified survey mode and functional mode categories can be used in any sequence that provides useful results; however, the general workflows for imaging typically begin with a survey mode to help orient the practitioner or sonographer to the anatomy being studied, and then follow with one or more functional modes. Moreover, survey modes can be repeated in a workflow, such as where it can be useful for the operator to obtain further definition of a particular location for subsequent functional mode imaging.

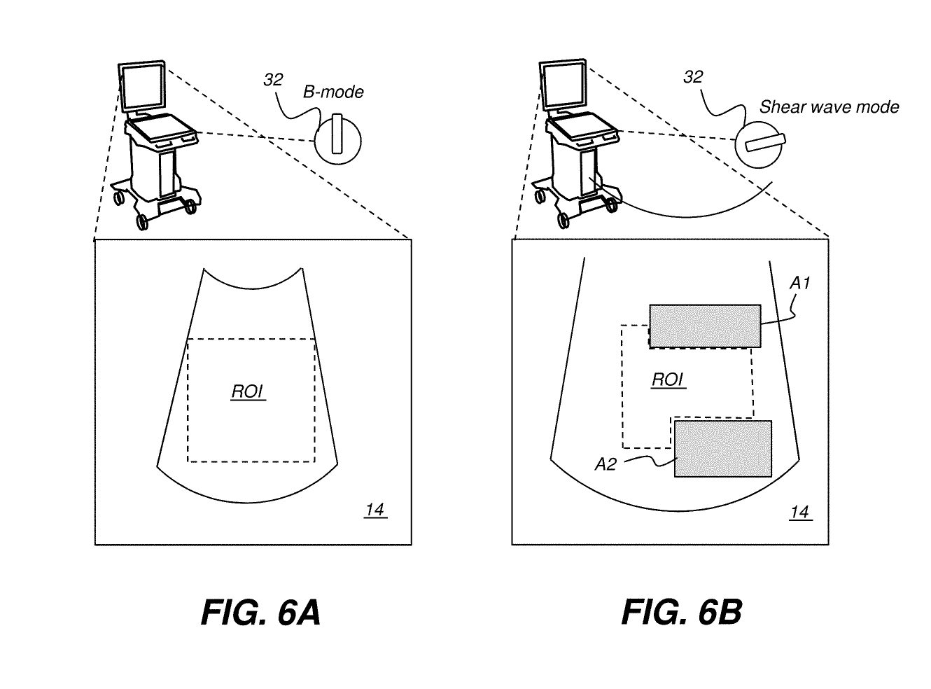

The schematic diagrams of FIGS. 6A and 6B show ultrasound system with display/monitor 14. FIG. 6A shows an ROI imaged in a survey mode such as B-mode ultrasound, detecting the ROI, such as a patient's liver. The schematic diagram of FIG. 6B then shows areas A1 and A2 that can contain anatomy portions that are not suitably imaged and can be considered to be incompatible with ultrasound imaging in a particular functional mode. This can include anatomy such as the liver capsule and other tissue and fluid components that do not allow accurate elastography measurement and that should be avoided when using shear wave imaging SWEI ultrasound mode. An operator control switch 32 allows switching between modes.

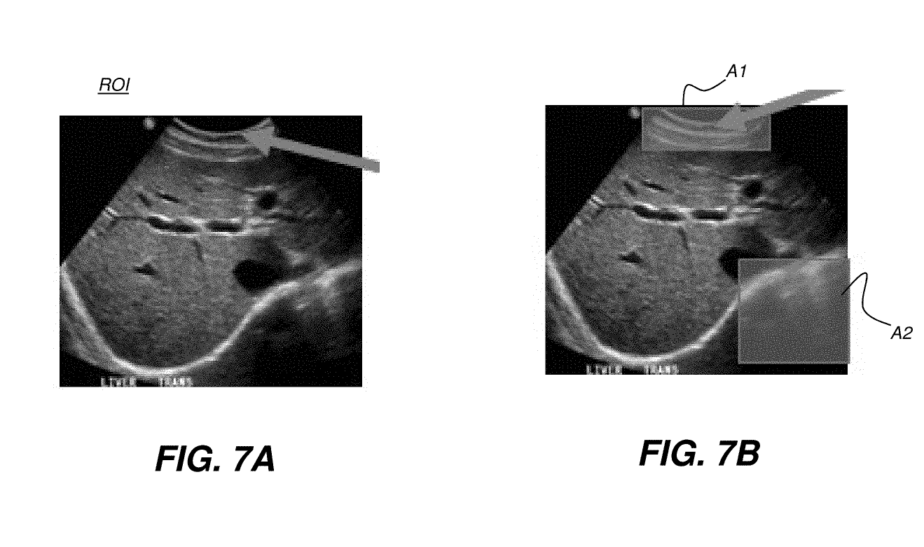

FIGS. 7A and 7B show, by way of example, an ROI containing the human liver and areas A1 and A2 considered not compatible and to be avoided when using SWEI imaging. These areas can include the liver capsule and other structures that make it difficult to properly sense and interpret the received acoustic signal, for example.

An embodiment of the present disclosure employs an approach using image processing, with detection and extraction of unique tissue features from ultrasound data, to identify locations suitable for elastography measurement and other locations that can be considered to lie outside of the range of shear wave measurement for elastography, that is, outside of the region in which a desired elastography measurement is possible. Using the method disclosed by the Applicants herein, unsuitable or incompatible locations, once identified, can be displayed to the Sonographer or other practitioner, for example, as graphical overlays, superimposed on the color display monitor of the ultrasound system. Such a graphical visualization related to tissue compatibility can provide a visual cue to the clinical ultrasound sonographer that these identified locations are out of range and not compatible for accurate shear wave elastography measurement. One or more portions of internal tissue considered unsuitable for a particular functional imaging type may or may not be overlapped by the modified region of interest. Incompatibility can be based on tissue response characteristics or on continuous or periodic fluid or gas movement, for example.

The disclosed system and method can be beneficial to practitioners, particularly to sonographers who may not otherwise be aware that specific anatomical regions otherwise imageable within the B-mode ROI are incompatible with, and should be avoided for, elastography measurement. This feature relates not only to liver elastography, as described above, but also to other applications using shear wave imaging or other functional imaging modes in which particular anatomy may include areas not measurable for the particular type of acoustic signal that is generated and to be avoided when using this operational mode. For SWEI imaging, areas not well suited to elastography measurement can include, for example, anatomy that includes larger regions of moving fluid, such as blood vessels and the like.

The method can be used to discourage the use of acoustical radiation to portions of the ROI that may not respond well to imaging when using particular functional imaging modes.

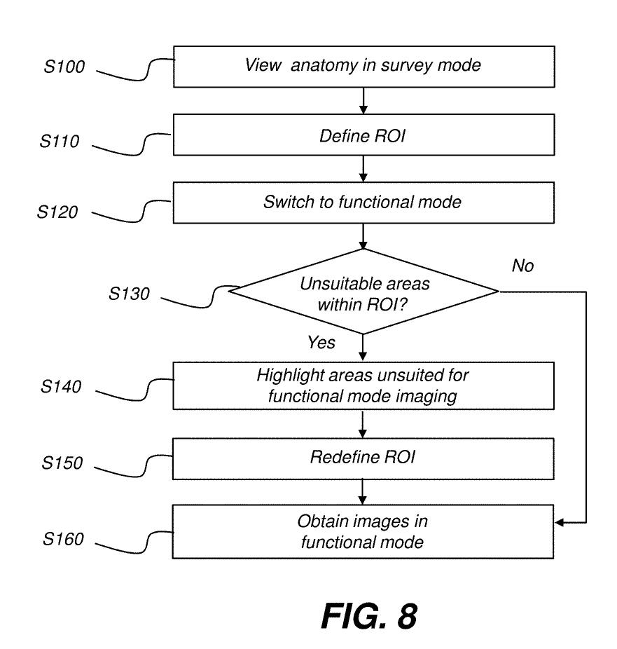

The logic flow diagram of FIG. 8 shows a typical sequence of steps for ultrasound imaging having automated ROI definition according to an embodiment of the present disclosure. In a survey mode imaging step S100, the patient anatomy is imaged using B-mode or other survey mode imaging procedure. In an ROI definition step S110, the displayed survey mode image is used to identify the overall ROI for subsequent measurement. In a functional mode selection step S120, the sonographer or practitioner switches to a functional mode for the particular exam. For the example described with reference to FIGS. 7A and 7B, this step involves switching from the survey mode (typically B-mode) to shear wave imaging mode. Alternately, step S120 can switch between different functional modes, such as first using a Doppler mode, then following with shear wave mode.

Continuing with the logic flow of FIG. 8, a test step S130 executes. In test step S130, system logic determines, from survey mode imaging data and, optionally, from predetermined workflow logic, whether or not the overall ROI defined in step S110 contains an area or areas that are not suitable for the functional mode selected in step S120. For the liver examination example shown in FIGS. 6A-7B, for example, the system determines that areas A1 (containing the liver capsule) and A2 (containing areas with perceptible blood or other fluid flow) are not well suited for imaging using the selected shear mode imaging signal. Based on test step S130 results, a highlight step S140 then analyzes image data from the survey mode ROI to identify and display one or more areas that are considered to be unsuitable for the selected functional mode. As in the example of FIG. 7B, the areas identified are highlighted by a box or outline or can be otherwise blocked from the ROI. An ROI redefinition step S150 then displays the redefined ROI accordingly, removing the identified areas from the overall ROI as described previously. This defines a revised ROI that excludes one or more areas as shown in the example of FIG. 6B. A measurement acquisition step S160 can then execute, obtaining and displaying ultrasound measurements using the same or different functional mode from step S120. Measurements can also be recorded, stored, and transmitted as part of step S160.

As shown in the FIG. 8 sequence, test step S130 may determine that there are no areas of the ROI that are unsuitable for the particular functional mode. In that case, operation goes directly to measurement acquisition step S160 for imaging and display.

Image analysis techniques for identifying tissue type and suitability for the selected functional mode can include any of a number of techniques, such as methods that identify morphology from stored patient models that have been generated according to a patient population. According to an embodiment of the present disclosure, anatomy data from a number of models are available for use, with the models indexed by patient attributes such as age, sex, weight, height, and other factors. Methods for identifying ROIs can include techniques that identify tissue types or anatomical locations using acoustical properties or other features of the imaged tissue. Prior knowledge of the patient anatomy and of established rules and recommendations for imaging constraints can also be used, referenced by the system software.

In addition to identifying areas not suitable for the selected functional imaging mode, the control software can also define blocked areas and actively constrain the acoustical signal emission over the identified anatomy. Thus, for example, with reference to FIG. 7B, the system can “lock out” emission or reception of acoustical energy over blocked areas A1 and A2. When the ultrasound transducer is positioned over a blocked area, the transducer, or an appropriate portion of the transducer, is prevented from emitting an acoustical signal or the signal is attenuated.

The described system and method could be configured as a software tool/package that is integrated with the core operating software of the ultrasound system. As such, when a shear wave elastography mode is launched in step S120 (FIG. 8), the software will be triggered to operate on the currently displayed image as the ROI. The software can be configured to analyze the currently displayed image, detect regions where the elastography measurements are not suitable, such as using the model data described previously, and highlight particular areas using a predefined color, tonal shade, or other setting. In addition, the ROI cursor can be limited from movement into the regions identified as unsuitable. As part of the system configuration and method of the present disclosure, visual markers, landmarks, or queues can be displayed that highlight the areas where shear wave imaging is not recommended, for example, due to the presence of particular anatomical structures where shear waves do not suitably propagate.

Similarly, visual markers, landmarks, or queues can be displayed that highlight the areas where the functional mode, such as Shear Wave imaging, is recommended in the presence of anatomical structures that are not well suited to the functional imaging type. Optionally, the region-of-interest indicator used for the measurement can move to the constrained anatomical areas.

Visual markings used for highlighting in step S140 (FIG. 8) can be transparent or opaque. These markings may overlay or overlap the displayed image to block out or cover areas not suitable for the functional imaging mode selected. The markers can alternatively be an outline of the unsuitable region or area. While perhaps not preferred by some practitioners, it is noted that, as an alternative, highlighting can be configured such that transparent visual markers are overlaid or overlap the displayed image to indicate the areas of the displayed image which are recommended for ultrasound/elastography imaging.

Applicants have described an image processing system and method employing extracting unique tissue features from ultrasound data to identify locations where a reliable elastography measurement may not be suitable, desirable, or even possible. Such locations once identified can be displayed as graphical overlays on the color display monitor of the ultrasound system. This provides a visual cue to the clinical ultrasound user that these locations should be avoided for a shear wave elastography measurement. This method can be of assistance to less experienced sonographers who may otherwise not be aware that specific anatomical regions should be avoided for an elastography measurement or other type of functional acoustic imaging. In the description above, the use of the present method is described for liver elastography; this method, however, can be applied to any shear wave application, such as for avoidance of blood vessels and the like.

An embodiment of the present disclosure can be implemented as a software program. Those skilled in the art will recognize that the equivalent of such software may also be constructed in hardware. Because image manipulation algorithms and systems are well known, the present description will be directed in particular to algorithms and systems forming part of, or cooperating more directly with, the method in accordance with the present invention. Other aspects of such algorithms and systems, and hardware and/or software for producing and otherwise processing the image signals involved therewith, not specifically shown or described herein may be selected from such systems, algorithms, components and elements known in the art.

A computer program product may include one or more storage medium, for example; magnetic storage media such as magnetic disk (such as a floppy disk) or magnetic tape; optical storage media such as optical disk, optical tape, or machine readable bar code; solid-state electronic storage devices such as random access memory (RAM), or read-only memory (ROM); or any other physical device or media employed to store a computer program having instructions for controlling one or more computers to practice the method according to the present invention.

The methods described above may be described with reference to a flowchart. Describing the methods by reference to a flowchart enables one skilled in the art to develop such programs, firmware, or hardware, including such instructions to carry out the methods on suitable computers, executing the instructions from computer—readable media. Similarly, the methods performed by the service computer programs, firmware, or hardware are also composed of computer—executable instructions.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim.

In the following claims, the terms “first,” “second,” and “third,” and the like, are used merely as labels, and are not intended to impose numerical requirements on their objects.

The invention has been described in detail with particular reference to a presently preferred embodiment, but it will be understood that variations and modifications can be effected within the spirit and scope of the invention. The presently disclosed embodiments are therefore considered in all respects to be illustrative and not restrictive. The scope of the invention is indicated by the appended claims, and all changes that come within the meaning and range of equivalents thereof are intended to be embraced therein.

1. A method comprising:
displaying an ultrasound image of subject tissue that defines a region of interest, the image acquired in a first measurement mode;
in response to an operator instruction:
(i) switching to a second measurement mode;
(ii) analyzing the subject tissue within the region of interest for compatibility with the second measurement mode; and
(iii) highlighting one or more areas of the subject tissue for removal from the region of interest and displaying a revised region of interest according to the mode compatibility analysis;
directing one or more signals for the second measurement mode to the revised region of interest; and
displaying, storing, or transmitting at least a portion of the measurement result.

2. The method of claim 1 wherein the first measurement mode is B-mode ultrasound imaging.

3. The method of claim 1 wherein the second measurement mode is a shear wave ultrasound imaging mode.

4. The method of claim 1 wherein highlighting the one or more areas of the subject tissue comprises displaying an outline.

5. The method of claim 1 further comprising recording, storing, or transmitting the measurement results.

6. The method of claim 1 wherein analyzing is performed by processor logic for an ultrasound system.

7. The method of claim 1 further comprising automatically constraining second measurement mode signals to the revised region of interest.

8. The method of claim 1 wherein at least one of the one or more areas of the subject tissue for removal image a liver capsule.

9. The method of claim 1 wherein at least one of the one or more areas of the subject tissue for removal image a blood vessel.

10. A method comprising:
displaying an ultrasound image of a region of interest of subject tissue for imaging in a shear wave imaging mode;
in response to an operator input, modifying the region of interest within the displayed ultrasound image by determining suitability for shear wave elastography imaging;
on the displayed ultrasound image, highlighting one or more portions of the region of interest determined to be unsuitable for elastography imaging and lying outside the modified region of interest;
performing an elastography measurement for the modified region of interest; and
displaying, storing, or transmitting at least a portion of the elastography measurement result.

11. The method of claim 10, wherein the determining unsuitable portions for elastography is accomplished automatically.

12. The method of claim 10 wherein the modified region of interest overlaps the one or more unsuitable portions.

13. The method of claim 10 wherein the modified region of interest does not overlap the one or more unsuitable portions.

14. A method comprising:
displaying an ultrasound image of subject tissue that defines a region of interest, the image acquired in a survey measurement mode;
in response to an operator instruction:
(i) switching to a functional measurement mode;
(ii) removing one or more portions of the subject tissue from the defined region of interest according to incompatibility of the one or more portions of subject tissue with the functional measurement mode; and
(iii) highlighting one or more of the removed portions of the subject tissue and displaying a revised region of interest according to the incompatibility;
directing one or more signals for the functional measurement mode to the revised region of interest; and
displaying, storing, or transmitting at least a portion of the measurement result.

15. The method of claim 14 wherein the survey measurement mode is an A-mode or B-mode.

16. The method of claim 14 wherein the functional measurement mode is a shear wave elasticity imaging mode.

17. The method of claim 14 further comprising blocking, from the one or more removed portions of the subject tissue, the one or more signals for the functional measurement.
